(19) United States
(12) Patent Application Publication
(10) Pub. No.: US 2010/0042058 A1
(43) Pub. Date: Feb. 18, 2010

(54) System and Method for Draining Bodily Fluids from a Treatment Site

(76) Inventor: Sheryl L. Casey, Bartow, FL (US)

Correspondence Address: Gray Robinson;ATTN: STEFAN V. STEIN/ IP DEPT., 201 N. Franklin Street, Suite 2200, Post Office Box 3324, TAMPA, FL, 33601-3324, US

(21) Appl. No.: 12/191,566

(22) Filed: Aug. 14, 2008

(51) Int. Cl. A61M 1/00

(52) U.S. Cl. 604/290; 604/315

(57) ABSTRACT

Disclosed is a drainage system that extracts fistula-produced discharge, or other bodily fluids, from an injury, such as a burn, laceration, or contusion. The system allows for the collection and convenient disposal of these fluids. The system components generally include an adhesive backed pad that is secured to a clear plastic bag and a length of perforated tubing that is slidably positioned within the bag. The tubing is connected to a suction pump and a collection reservoir. The pump generates a suction along a perforated extent of the tubing to thereby evacuate bodily fluids that collect within the bag.

### FIELD OF THE INVENTION

This invention relates to a drainage system for a patient. More particularly, the invention relates to a sealed enclosure for creating a vacuum over an area being treated to thereby drain away any unwanted fluids.

### DESCRIPTION OF THE BACKGROUND ART

The use of suction in the treatment of wounds is known in the art. An example of this is disclosed U.S. Pat. No. 7,198,046 to Argenta. Argenta discloses a wound treatment that employs a reduced pressure. Negative pressure is created over the wound to promote tissue migration and facilitate closure. The treatment utilizes a fluid impermeable cover that is sealed around the site of the wound. An open-celled foam screen is also placed beneath the cover but over the wound.

Likewise, U.S. Pat. No. 7,381,859 to Hunt discloses a removable wound closure that employs negative pressure. The negative pressure is applied to promote healing and to limit the formation of adhesions. The dressing of Hunt includes a layer of a porous foam material that is enclosed by sheets of elastomeric material. An additional elastomeric sheet covers the entire wound dressing and is sealed to the skin surrounding the wound. The elastomeric sheets are punctuated by a number of holes. Fluids are drawn by negative pressure through the holes and the foam via a vacuum device and a suction tube.

Although the inventions of the prior art each achieve their specific individual objectives, they all suffer from common drawbacks. Namely, wound dressings of the prior art are generally of a single size and are not easily configurable to fit wounds of different sizes or shapes. This means that different dressing must be provided for use with different types of wounds. Moreover, the dressings of the prior art do not permit the amount or location of the suction to be changed. As a result, the dressings of the prior art often do not utilize the suction in the most efficient manner. The system of the present invention is aimed at overcoming these shortcomings.

### SUMMARY OF THE INVENTION

It is therefore an object of this invention to provide a vacuum drainage system that can be configured to fit treatment sites of varying size and shape.

It is another object of this invention to provide a vacuum drainage system that utilizes a slidable length of perforated tubing to permit the suction created thereby to be selectively positionable in and around a treatment site.

It is yet another object of this invention to provide a drainage system that includes a length of perforated tubing that can be cut to vary the amount of suction created.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a top plan view of the drainage system of the present invention.

FIG. 2 is a side elevational view of the drainage system of the present invention.

FIG. 3A is a bottom plan view of the pad of the drainage system showing the release liner being removed.

FIG. 3B is an additional bottom plan view of the pad.

FIG. 4 is a perspective view of the drainage system showing one of the optional perforated areas of the pad being removed.

FIG. 5 is a perspective view showing the base of the drainage system being cut away.

FIG. 6 is a perspective view showing the perforated extent of the tubing being cut.

FIG. 7 is a perspective view showing the tubing being slidably positioned within the bag.

FIG. 8 is a perspective view showing the system in place on a patient.

Similar reference characters refer to similar parts throughout the several views of the drawings.

### DETAILED DESCRIPTION OF THE PREFERRED EMBODIMENT

The present invention relates to drainage system that extracts bodily fluids from a patient. These fluids can be, for example, fluids from a wound, burn, laceration, or contusion. The fluids can also include fistula-produced discharge, blood, fecal matter, urine, or any other bodily fluids that must be evacuated in order to properly treat or care for a patient. The system allows for the collection and convenient disposal of these bodily fluids.

The system components generally include an adhesive backed pad that is secured to a clear plastic bag and a length of perforated tubing that is slidably positioned within the bag. The tubing is connected to a suction pump and a collection reservoir. The pump generates a suction along a perforated extent of the tubing to thereby evacuate bodily fluids that collect within the bag.

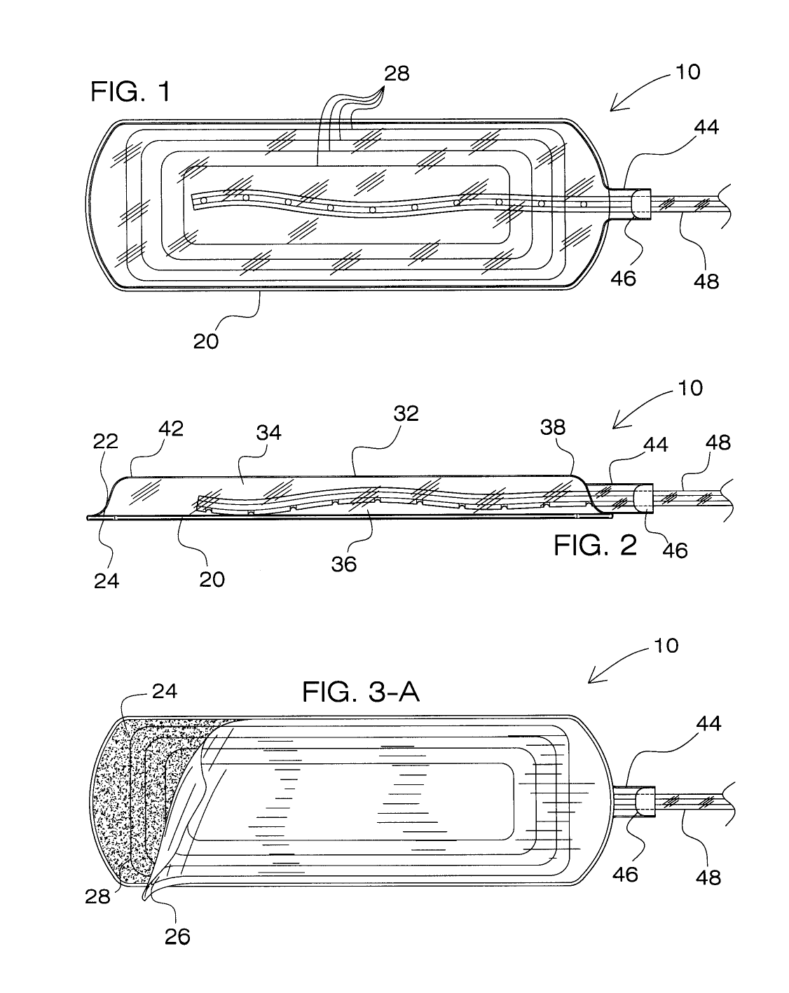

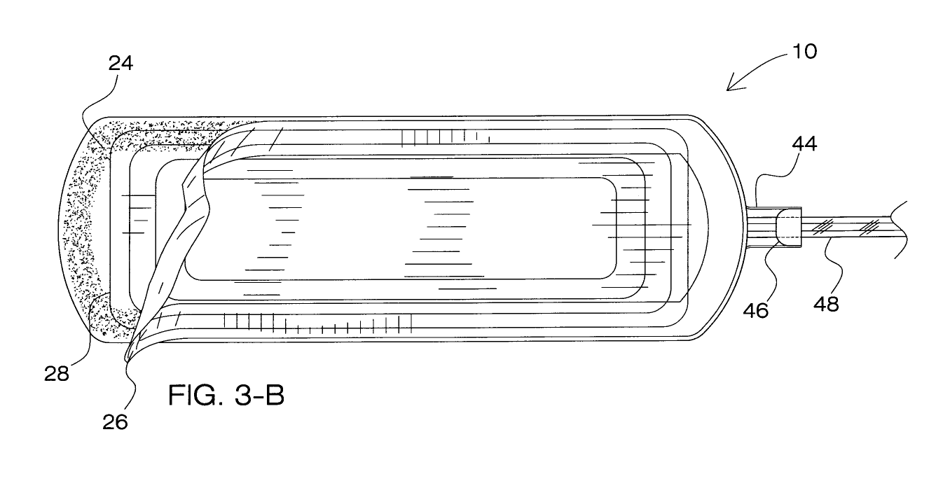

As noted in FIGS. 1 and 2, the base of the system 10 is an adhesive backed pad 20. Pad 20 is designed to be adhered around the site being treated for prolonged periods of time via an adhesive backing. Accordingly, pad 20 is preferably made from a soft pliant material, such as a synthetic rubber or an elastomeric. A hydrocolloid pad can optionally be used. Whatever material is used, pad 20 is defined by upper and lower surfaces (22 and 24), with lower surface 22 being coated with a medical grade, non-permanent adhesive. A release liner 26 is positioned over the adhesive and serves to preserve the adhesive prior to the pad being secured to the skin. Release liner 26 also prevents the lower surface 24 of pad 20 from otherwise becoming contaminated.

Pad 20 is designed to be positioned over and around a treatment site from which bodily fluids are to be evacuated. By way of non limiting example, the treatment site can be a wound, laceration, contusion, burn, or a bodily orifice, such as the anus. Prior the application of pad 20, a portion of pad 20 is cut out to fit about the site being treated. FIG. 5 illustrates a scalpel being used to cut out a suitable opening in pad 20. In such an embodiment, the care giver simply cuts out a portion of the pad depending upon the size and location of the site being treated, and on the contour of the patient, and on the location of skin folds.

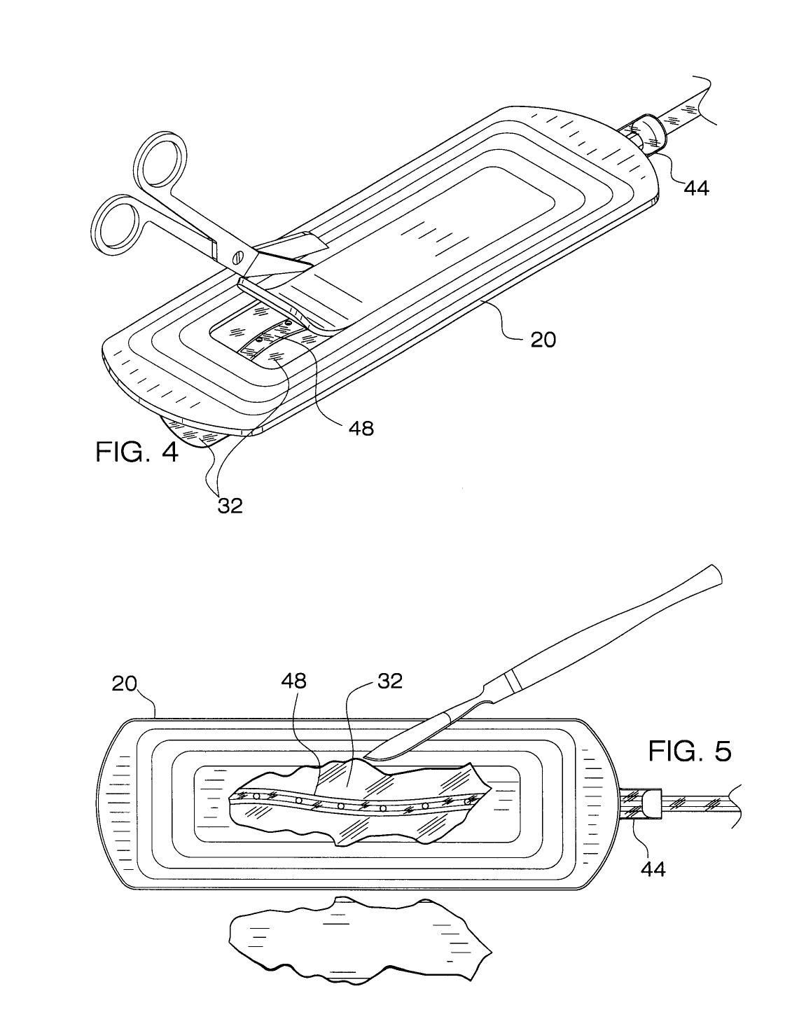

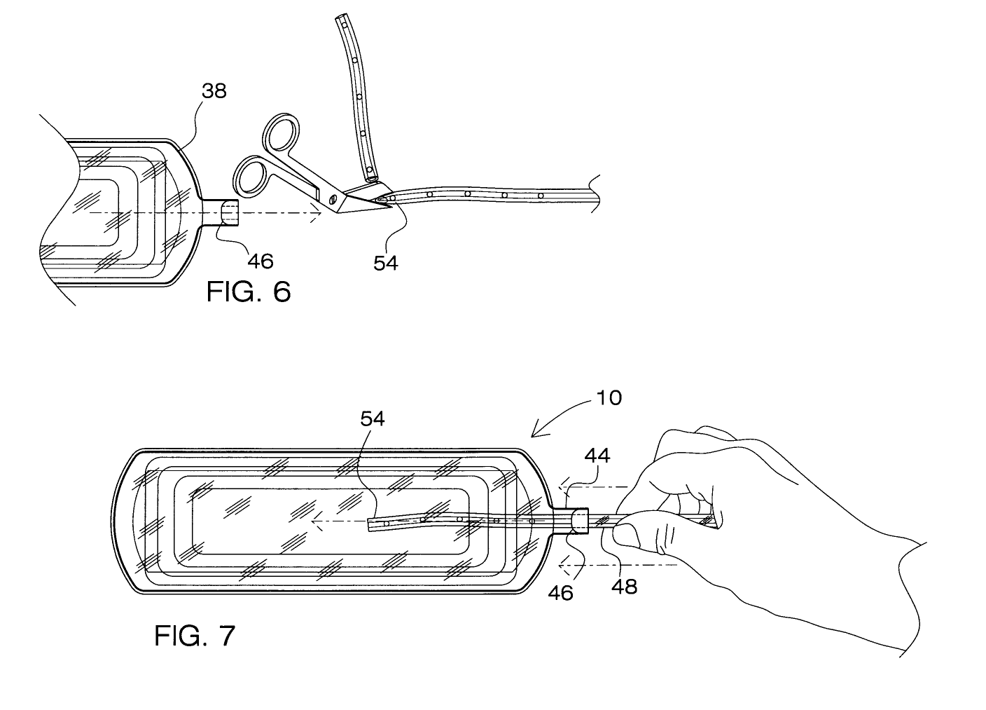

As an optional alternative, and as noted in FIG. 4, a series of perforated cut outs 28 can be formed through the pad. The cut outs 28 allow a portion of pad 20 to be removed as needed to accommodate the site. In the depicted embodiment, four concentric cut outs 28 are provided. In order to orient pad 20 over larger sites, all four cut outs 28 can be removed. Conversely, for smaller sites, only the innermost cutout 28 is removed. It should be noted, however, that the use of cut outs, perforated or otherwise, is an optional feature of the invention. Furthermore, although four cut outs are depicted, the use of other numbers of cut outs is also within the scope of the invention.

The upper surface 22 of pad 20 is designed to support a collection bag 32. As illustrated in FIG. 2, bag 32 is defined by a closed upper extent 34, an opened lower extent 36, and proximal and distal ends (38 and 42). Bag 32 is secured to pad 20 by sealing the peripheral edge of the bag's opened lower extent 36 to the upper surface 22 of pad 20. Bag 32 is designed to collect the discharge from the site and to otherwise create an environment to promote healing. In the preferred embodiment, a clear bag made from a medical grade plastic is used. Fluids are evacuated from bag 32 via a port 44 that is formed within the proximal end 38 of bag 32 (note FIG. 2). Port 44 houses an internal seal 46 that allows tubing 48 to extend into the interior of bag 32. By way of seal 46, an airtight fit is formed around tubing 48. The fit also prevents contaminants from destroying the sterile environment that initially exists within bag 32.

A length of tubing 48 is employed in removing any bodily fluids that are collected in bag 32. Any of a wide variety of medical grade tubings will suffice. Tubing 48 is generally defined by proximal, distal, and intermediate extents (52, 54, and 56, respectively). As illustrated in FIG. 7, distal extent 54 of tubing 48 is perforated. Additionally, intermediate extent 56 of tubing 48 can be slid into or out of port 44. This slidable fit allows a caregiver to properly position the perforated extent 54 of tubing 48 within bag 32. More specifically, a caregiver can selectively position the perforated extent 54 such that it overlies the site being treated. This ensures proper drainage of the site and an efficient use of the suction. In the alternative, in the event large volumes of fluid and/or fecal matter need to be evacuated, the tubing 48 can be cut to eliminate the perforated extent 54 altogether. In such an embodiment, the end of tubing 48 is simply secured to port 44 in a fluid tight manner. This arrangement may be employed, for example, if the system is applied over the anus of a patient.

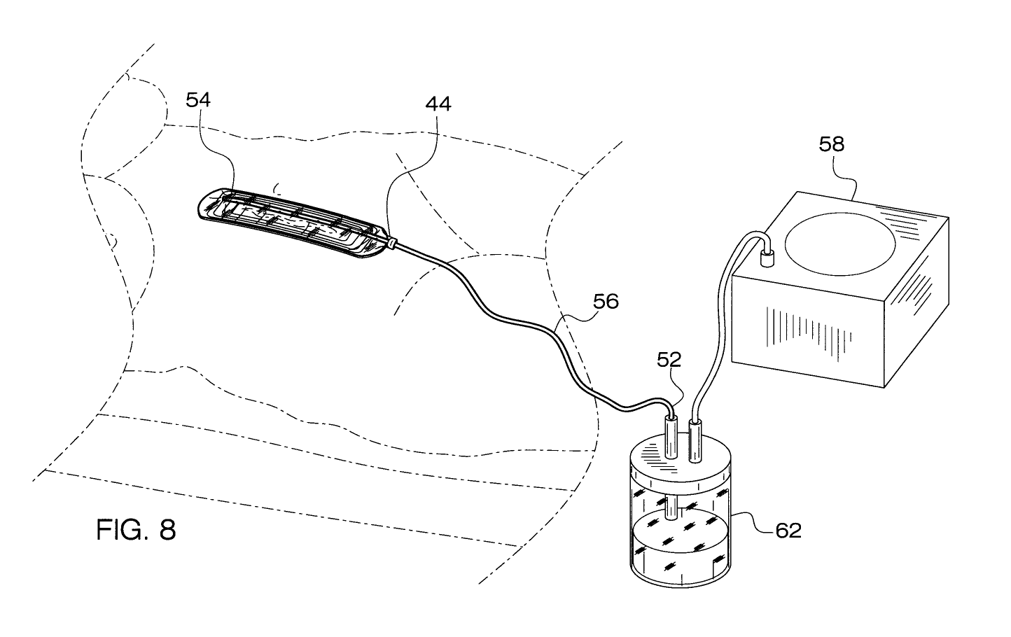

As illustrated in FIG. 8, a pump 58 and a reservoir 62 are secured to the proximal end 52 of tubing 48. Pump 58 draws a vacuum to thereby create suction along the perforated extent 54 of tubing 48. Reservoir 62 is used to collect the bodily fluids extracted by way of tubing 48.

In use, a caregiver first examines the treatment site to ascertain its shape and size. A corresponding opening is then formed within the pad 20. This can be accomplished by either cutting an opening into the pad or removing one or more inserts 28. Release liner 26 is then removed from the remaining peripheral extent of pad 20. Pad 20 is then adhered around the site. During this step, the caregiver must make sure that the site is positioned within the opening so as to ensure proper drainage.

Next, depending upon the size and shape of the site, the caregiver can cut the perforated extent 54 of tubing 48. For larger sites, a longer perforated extent 54 is necessary. For smaller sites, a shorter perforated extent 54 is necessary. The properly tailored perforated extent 54 is thereafter inserted into bag 32 by way of the port 44 and seal 46. During this step, the caregiver inserts tubing 48 and otherwise positions it as needed to place the perforated extent 54 over the site being treated. The proper placement of perforated extent 54 ensures that all of the fluids are evacuated.

The suction pump 58 is thereafter activated, which draws a vacuum within tubing 48 and at perforations. As a result, fluids from the site are collected by the perforations and are transported by tubing 48 into the collection reservoir 62. Reservoir 62 can thereafter be empted as needed. The use of the system eliminates the need for caregivers to repeatedly change dressings or otherwise clean the site. The need to repeatedly change dressings is of particular concern with fistulas, which continually emanate bodily fluids over prolonged periods. Suction pump 58 also serves to create a slight vacuum within collection bag 32. This, in turn, causes collection bag 32 to adhere to the patient's skin, thereby creating a hermetic seal. Use of the inventive system also protects the skin from breakdown, reduces odor, contains the drainage, enables the accurate measurement of fluid output, and further permits the caregiver to visualize fluid output. All of this is achieved in a cost effective manner that promotes comfort for the patient and decreases the treatment time of the caregiver.

The use of a clear plastic bag 32 allows the wound site to be visually inspected throughout the course of treatment. If the shape and or size of the wound changes over time, the caregiver can remove tubing 48 and cut the perforated extent 54. The caregiver can also reposition tubing 48 to ensure that it remains over the areas of the treatment site that are secreting the most fluids. Both steps ensure the efficient removal of fluids.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A drainage system for a treatment site comprising:
a hydrocolloid pad having a lower adhesive surface, an upper surface, a series of concentrically oriented perforated cut outs formed through the pad, a release liner secured to the lower adhesive surface;
a clear plastic bag having a closed upper extent, an opened lower extent with a peripheral edge, and proximal and distal ends, a port with a seal formed within the proximal end of the bag, the peripheral edge of the bag being secured to the upper surface of the pad;
a length of tubing having a proximal extent, a distal extent and an intermediate extent therebetween, a series of perforations formed within the distal extent and defining a perforated extent, the intermediate extent being slidably secured within the port and seal such that the perforated extent is within the plastic bag, wherein the seal forms an air tight barrier between the tubing and the bag;
a pump and a reservoir secured to the proximal end of the tubing, wherein the pump draws a vacuum to thereby create suction along the perforated extent of the tubing;
whereby a user can vary the length of the perforated extent and the amount of suction created thereby by cutting the tubing, and whereby the user can selectively remove one or more of the perforated cut outs to accommodate different sized treatment sites.

2. A drainage system comprising:
a pad having a lower surface and an upper surface, the pad designed to be secured to a patient's skin;
a collection bag secured to pad, a port formed within the bag;
a length of tubing having a proximal extent, a distal extent and an intermediate extent therebetween, a series of perforations formed within the distal extent and defining a perforated extent, the intermediate extent being slidably secured within the port such that the perforated extent is within the collection bag;
a pump secured to the proximal end of the tubing, wherein the pump draws a vacuum to thereby create suction along the perforated extent of the tubing;
whereby a user can vary the length of the perforated extent and the amount of suction created thereby by cutting the tubing.

3. The system as described in claim 2 wherein the pad is a hydrocolloid pad.

4. The system as described in claim 2 wherein an airtight seal is formed between the tubing and the port.

5. The system as described in claim 2 wherein a series of perforated cut outs are formed through the pad.

6. The system as described in claim 5 wherein the cut outs are concentrically oriented.

7. A method of draining a treatment site on a patient, the method utilizing a clear plastic bag that is secured to an adhesive pad, with a length of tubing being slidably positioned within the bag by way of a sealed port, the tubing having a perforated extent, the method compromising the following steps:
determining the size of the treatment site and forming a cut out within the adhesive pad to accommodate the site;
securing the adhesive pad to the patient while ensuring that the site is positioned within the cut out;
inserting or withdrawing the tubing from the bag as necessary to position the perforated extent over the site;
drawing a vacuum at the end of the tubing to create suction along the perforated extent and thereby drain fluids from the bag;
whereby the position and degree of suction can be selectively changed by removing the tubing from the bag, cutting the perforated extent to reduce its length, and inserting the tubing back into the bag.
